(19) United States
(12) Patent Application Publication
(10) Pub. No.: US 2022/0152399 A1
(43) Pub. Date: May 19, 2022

(54) DEEP BRAIN STIMULATION SYSTEM WITH AMPLITUDE-MODULATED TEMPORAL PATTERNS

(71) Applicant: University of Connecticut, Farmington, CT (US)

(72) Inventors: Martin Han, Mansfield, CT (US); Sabato Santaniello, Vernon, CT (US)

(21) Appl. No.: 17/438,856

(22) PCT Filed: Mar. 13, 2020
(86) PCT No.: PCT/US2020/022726

§ 371 Date: Sep. 13, 2021

Related U.S. Application Data
(60) Provisional application No. 62/818,649, filed on Mar. 14, 2019.

(51) Int. Cl. A61N 1/36; A61N 1/05

(52) U.S. Cl. CPC A61N 1/36171; A61N 1/0534; A61N 1/36192; A61N 1/36139; A61N 1/36064; A61N 1/36096; A61N 1/36132; A61N 1/36067

(57) ABSTRACT

A deep brain stimulation (DBS) system provides for treating a human brain exhibiting a pathological condition. A controller generates a signal waveform defining a series of DBS pulses within an amplitude modulation envelope. The frequency of the amplitude modulation envelope may be less than or equal to half of a frequency of the DBS pulses. A driver circuit generates an amplified, amplitude-modulated electrical waveform corresponding to the signal waveform. A DBS electrode electrically coupled to the driver circuit converts the electrical waveform to a stimulation waveform to entrain neurons of the deep brain tissue in a normal oscillation pattern, thereby suppressing the pathological oscillation pattern to treat the pathological condition.

### RELATED APPLICATIONS

This application claims the benefit of U.S. Provisional Application No. 62/818,649, filed on Mar. 14, 2019. The entire teachings of the above application are incorporated herein by reference.

### GOVERNMENT SUPPORT

This invention was made with government support under grant no. DC014044 from the National Institutes of Health. The government has certain rights in the invention.

### BACKGROUND

Deep Brain Stimulation (DBS) is a therapy for the treatment of movement disorders, obsessive-compulsive disorders, depression, and post-traumatic disorders via electrical stimulation of the brain. Despite being clinically used, DBS remains highly inefficient and prone to side-effects. Inefficiency and side-effects can stem from improper stimulation or overstimulation of brain tissue, which leads to fast deterioration of the hardware used to deliver the electrical stimulation, costly maintenance, and frequent surgical replacements.

### SUMMARY

Example embodiments provide DBS systems and methods for treating a human brain exhibiting a pathological condition. In one embodiment, a DBS system comprises a controller, a driver circuit, and at least one DBS electrode. The controller may be configured to generate a signal waveform defining a series of DBS pulses within an amplitude modulation envelope. A frequency of the amplitude modulation envelope may be less than or equal to half of a frequency of the DB S pulses. The driver circuit may include an input port and an output port and may amplify the signal waveform received from the controller via the input port to produce an electrical waveform available at the output port. The DBS electrode may be electrically coupled to the output port of the driver circuit and configured to convert the electrical waveform to a stimulation waveform to entrain neurons of the deep brain tissue in a normal (e.g., common or nonpathological) oscillation pattern, thereby suppressing the pathological oscillation pattern to treat the pathological condition.

In some embodiments, the DBS system comprises at least one DBS electrode that is configured to be implanted in electrical communication with deep brain tissue within a brain of the patient. The deep brain tissue is known to exhibit a pathological oscillation pattern related to the pathological condition. The region of deep brain tissue is selected from a group consisting of: the hippocampus, amygdala, medial prefrontal cortex, orbitofrontal cortex, anterior cingulate cortex, striatum, including the putamen and the caudate nucleus, thalamus, including the anterior, ventrointermediate, ventrolateral and dorsolateral thalamus, basal ganglia, fornix, dentate nucleus or substantia nigra. In some embodiments, the controller adjusts the signal waveform with the amplitude modulation envelope having characteristics that cause the at least one DBS electrode to produce a stimulation waveform specific to the respective region of deep brain tissue.

In some embodiments, the controller stores parameters for generating the signal waveform that results in the stimulation waveform having characteristics that treat neural pathological conditions. The neural pathological conditions include Parkinson's disease (PD), essential tremor (ET), obsessive compulsive disorder (OCD), depression, epilepsy, or psychiatric disorders such as post-traumatic stress disorder (PTSD). The controller can generate the amplitude modulation envelope at an amplitude and frequency that entrains the respective neurons of the region of deep brain tissue, at which the at least one DBS electrode is located, in the normal oscillation pattern in the region of deep brain tissue at which the at least one DBS electrode is located.

In some embodiments, the controller adjusts the amplitude modulation envelope such that the maximum modulation of the DBS pulse amplitude ranges from about 1 percent to about 100 percent of the maximum allowed pulse amplitude. The maximum modulation is referred to as “depth” hereafter in this application. In some embodiments, the controller generates DBS pulses with a frequency from about 50 Hertz to about 10,000 Hertz and generates the amplitude modulation envelope with a frequency from about 1 Hertz to about 5,000 Hertz.

The controller enables a clinician or the patient to adjust stimulation programming depending on feedback of the patient's reaction to the stimulation waveform. The controller adjusts the amplitude modulation envelope based on a user input.

In some embodiments, the DBS system further comprises at least one sensor that is communicatively coupled to the controller. The at least one sensor includes one of the following sensor types: an electro-chemical sensor, motion sensor, optical sensor, accelerometer, chemical, electrical, ultrasonic, piezoelectric, and imaging device. The at least one sensor is configured to be disposed in or at the patient at a location to observe an expression of an affectation that is symptomatic of the pathological condition. That location is selected from a group consisting of the deep brain tissue at which the at least one DBS electrode is located, a location elsewhere within the patient or a location at which movement of the patient is observable. In some embodiments, the sensor communicatively coupled to the controller provides data from the sensor, or information based on the data, to a clinician via an interface. In some embodiments, the controller adjusts a parameter of the amplitude modulation envelope as a function of the data received from the at least one sensor. In some embodiments, the sensor is further configured to be coupled to an appendage or head of the patient, offset from the patient with an orientation to observe motion of the patient, motion of an appendage of the patient, motion of a head of the patient, or offset from the patient with an orientation to detect eye gaze or eye saccades of the patient. In some embodiments, the location elsewhere within the patient is a subcutaneous, epidural, subdural, intracortical, or subcortical region of the patient.

In some embodiments, the driver circuit of the DBS system operates as a stimulation pattern generator that generates voltage waveform and converts the signal waveform to a current-controlled electrical waveform.

An example embodiment of the invention focuses on developing novel electrical stimulation patterns for use in DBS applications. These temporal patterns modulate the amplitude of electrical stimuli over time per pre-defined profiles. Thus, the embodiment may be leveraged in existing DBS systems or implemented in new systems/hardware, and, thus, may potentially encompass computer-implemented method, software, and hardware.

Embodiments that employ amplitude-modulated DBS (AM-DBS) include pulses that have the same shape and duration, and are equally spaced, while the pulse amplitude varies in time according to a pre-selected rule that is programmed in the pulse generator. For example, the pulse amplitude Aₖ of the generic k-th pulse at the generic time tₖ may vary according to the rule. Embodiments that employ AM-DBS may entrain neurons in a common oscillation at the frequency f of the envelope function, which will support the suppression of ongoing pathological oscillations that are prevalent in patients with neurological diseases (e.g., Parkinson's Disease [PD]) and that are also well-correlated with clinical symptoms.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 illustrates a deep brain stimulation (DBS) system in an example embodiment.

FIG. 2 is a block diagram of the DBS system in further detail.

FIG. 3A-C are timing diagrams illustrating waveform profiles that may be implemented in example embodiments.

FIG. 4 is a flow diagram illustrating a process of implementing DBS in one embodiment.

FIG. 5 is a diagram illustrating generation of a signal waveform in one embodiment.

FIG. 6 is a diagram illustrating user adjustment of a signal waveform in one embodiment.

FIG. 7 illustrates a function lookup table in one embodiment.

FIG. 8 illustrates amplitude-modulated pulses in one embodiment.

FIG. 9 is a diagram illustrating adjustment of a signal waveform based on biofeedback in one embodiment.

FIG. 10 is a diagram of DBS system in a further embodiment.

FIG. 11 illustrates a user interface in one embodiment.

### DETAILED DESCRIPTION

Example embodiments provide a system and method for safe, effective and efficient deep brain stimulation (DBS), serving as a means for successful treatment of movement disorders, obsessive-compulsive disorders, depression, epilepsy, and psychiatric disorders via electrical stimulation. A description of example embodiments follows.

### Definitions

“Entrain” describes a process of aligning of neuronal activity to the temporal structure of external rhythmic input streams. Entrainment usually entails phase alignment of brain oscillations (phase entrainment), but can also present as the alignment of rhythmically generated oscillatory events or bursts.

“Normal oscillation” describes normal rhythmic and/or repetitive electrical activity generated spontaneously and in response to stimuli by neural tissue in the central nervous system, which play a significant role in sensory-cognitive processes. Oscillatory electrical activity occurs in the brain when groups of neurons synchronize their firing activity. The oscillations are classified according to the neuron firing frequency. Alpha oscillations are relatively slow (8-13 Hz) and are associated with closed-eye relaxation; beta oscillations (13 to about 35 Hz) are commonly associated with normal waking consciousness, and motor planning; gamma oscillations (35-70 Hz) are associated with various aspects of perception, movement execution, and consciousness.

“Pathological oscillation” is an aberrant version of a common oscillation, such as, for example, the spike and wave oscillation, which is typical of generalized or absence epileptic seizures, and which resembles normal sleep spindle oscillations. Pathological oscillations are also evident in other movement disorders, as well as neuropsychiatric diseases

“Electrode” is an intracortical device designed to deliver electrical current to the brain or next to a neuron of interest and current is applied at a fixed frequency and time. The electrode elicits action potentials by changing the extracellular environment such that the voltage-gated ion channels open, depolarizing the neuron. Applying too much current can kill nearby cells. In some embodiments, these devices have an ultrafine tapered tip that can be inserted into individual biological cells, and the tips of these devices must be small with respect to the dimensions of the biological cell to avoid cell damage.

“Pathological condition” is a neuropathology that can include genetic disorders, congenital abnormalities or disorders, infections, lifestyle, or environmental health problems including malnutrition, and brain injury, spinal cord injury, nerve, injury, gluten sensitivity and neurochemical imbalances.

“Tissue” is an aggregate of neurons and oligodendrocytes forming a structural material with a specific function, in the nervous system of a multicellular organism.

“Programming phase” refers to a duration in which an example embodiment configures a combination of amplitude (in volts milliamps, depending on the device), frequency (in hertz), and/or pulse width (in microseconds) of the electrical pulses to be delivered by the neural stimulator. A trained physician may be tasked with implementing one or a combination of several programs in a patient exhibiting a pathological condition that optimizes the trade-off between clinical gain (i.e. how much the patient's disorder(s) improve) and side-effects secondary to overstimulation. The programming phase may occur before implantation of the deep brain stimulation device. The pre-programmed frequency of the pulses can be manipulated and may be chosen by the trained physician during the programming phase.

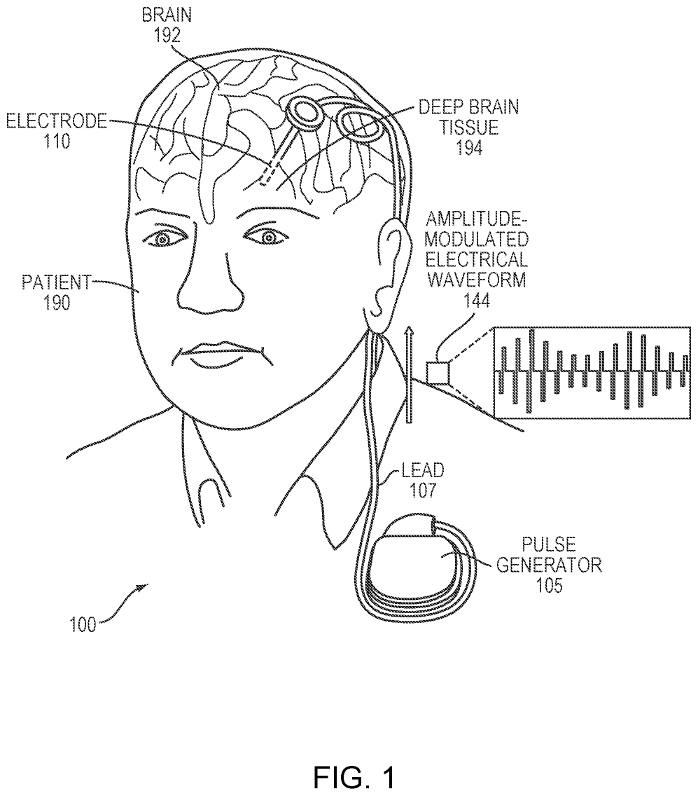

FIG. 1 depicts a deep brain stimulation (DBS) system 100 in an example embodiment. The system 100 may be partially or fully implanted within a patient 190 to deliver a DBS therapy to the patient 190. The system 100 includes a pulse generator 105 (e.g., a neurostimulator) configured to determine an amplitude-modulated signal waveform defining a series of DBS electric pulses and output a corresponding amplitude-modulated electrical waveform 144. A lead 107 (e.g., a wire or other conductive channel) may carry the electrical waveform 144 to a DBS electrode 110 that is implanted in electrical communication with the brain 192 of the patient 190. In particular, the electrode 110 may be implanted in electrical communication with deep brain tissue 194 within the brain 192 of the patient 190, and may extend to a depth of about 1 cm to about 15 cm within the brain 192. The DBS electrode 110 may be implanted in a region of the deep brain tissue 194 such as the hippocampus, amygdala, medial prefrontal cortex, orbitofrontal cortex, anterior cingulate cortex, striatum, including the putamen and the caudate nucleus, thalamus, including the anterior, ventrointermediate, ventrolateral and dorsolateral thalamus, basal ganglia, dentate nucleus, fornix, or substantia nigra. This deep brain tissue 194 of the patient 190 may have been previously determined to exhibit a pathological oscillation pattern related to a pathological condition exhibited by the patient 190. Although FIG. 1 illustrates a single-shank DBS electrode 110, the electrode 110 may include multiple shanks, or may include multiple discrete electrodes. The DBS electrode 110 may convert the electrical waveform 144 of the pulse generator 105 to a stimulation waveform to entrain neurons of the deep brain tissue 194 in a common oscillation pattern, thereby suppressing the pathological oscillation pattern to treat the pathological condition.

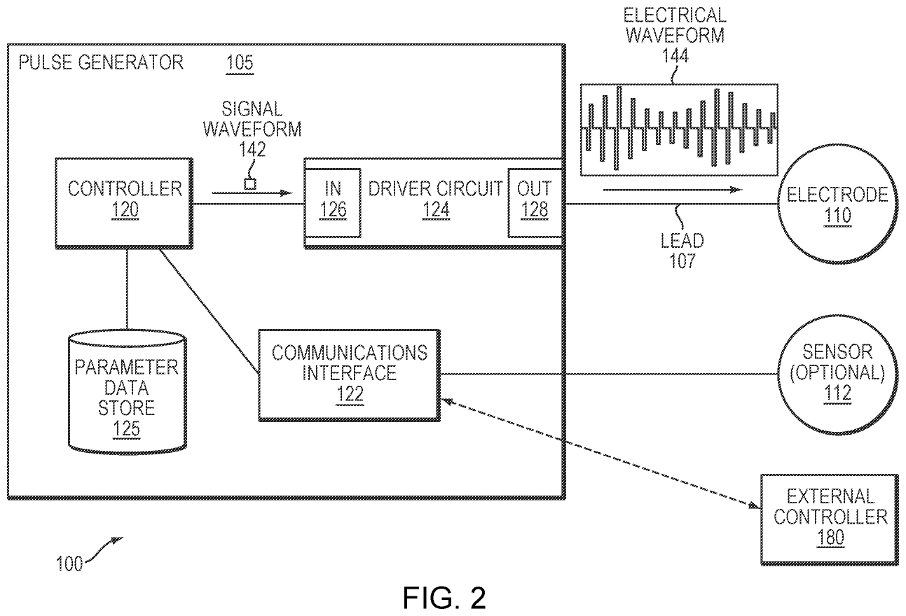

FIG. 2 illustrates the DBS system 100 in further detail. The pulse generator 105 may include a controller 120 configured to generate a signal waveform 142 defining a series of DBS pulses. A driver circuit 124 may receive the signal waveform 142 to an input port 126 and may amplify and convert the signal waveform 142 to produce the electrical waveform 144 (e.g., an amplitude-modulated waveform) at an output port 128. The driver circuit 124 may operate as a stimulation pattern generator that generates voltage waveform and converts the signal waveform 142 to a current-controlled electrical waveform 144. If the signal waveform 142 is digital, the driver circuit 124 may include a digital analog converter (DAC) to convert the digital signal waveform to the electrical waveform 144 as an analog signal output. The driver circuit 124 may also output the electrical waveform 144 as charge-balanced biphasic current pulses of variable amplitudes and durations to the electrode 110. The lead 107 may carry the electrical waveform 144 to the electrode 110, which may convert the electrical waveform 144 to a stimulation waveform to entrain neurons of the deep brain tissue 194 of the patient 190 in a normal oscillation pattern, thereby suppressing the pathological oscillation pattern to treat the patient's pathological condition. In further embodiments, the electrode 110 may be an array of multiple electrodes that are implanted at different locations of the deep brain tissue 194.

The pulse generator 105 may further include a parameter data store 125, which may include a storage medium (e.g., non-volatile computer memory) to store parameters for a DBS process as described further below. A communications interface 122 may include one or more interfaces for communications external to the pulse generator 105. For example, the communications interface 122 may include a wireless network interface for communicating wirelessly with an external controller 180. The communications interface 122 may also include circuit components that can detect voltages or other biosignals, such as differential amplifier, ADC converter, before such signals are routed to the controller 120. The external controller 180 may include a remote controller (e.g., a touchscreen or push-button device), a computer workstation such as a laptop, or a tablet or mobile computing device, and may be configured to enable user selection and monitoring of a DBS operation. A sensor 112 may also be communicatively coupled to the controller 120 via the communications interface 122 or another interface. The sensor 112 may include one or more individual sensing elements, which may include an electro-chemical sensor, motion sensor, optical sensor, accelerometer, chemical, electrical, ultrasonic, piezoelectric, and/or an imaging device. The sensor 112 may be disposed in or at the patient 190 at a location to observe an expression of an affectation that is symptomatic of the pathological condition. For example, the sensor 112 may be located at the same region of deep brain tissue at which at least one of the DBS electrode 110 is located, a location elsewhere within the patient (e.g., a subcutaneous, epidural, subdural, intracortical, or subcortical region of the patient), or a location at which movement of the patient 190 can be observed. In particular, the sensor 112 may be coupled to an appendage or head of the patient 190, offset from the patient 190 with an orientation to observe motion of the patient, motion of an appendage of the patient 190, motion of a head of the patient 190, or offset from the patient 190 with an orientation to detect eye gaze or eye saccades of the patient 190.

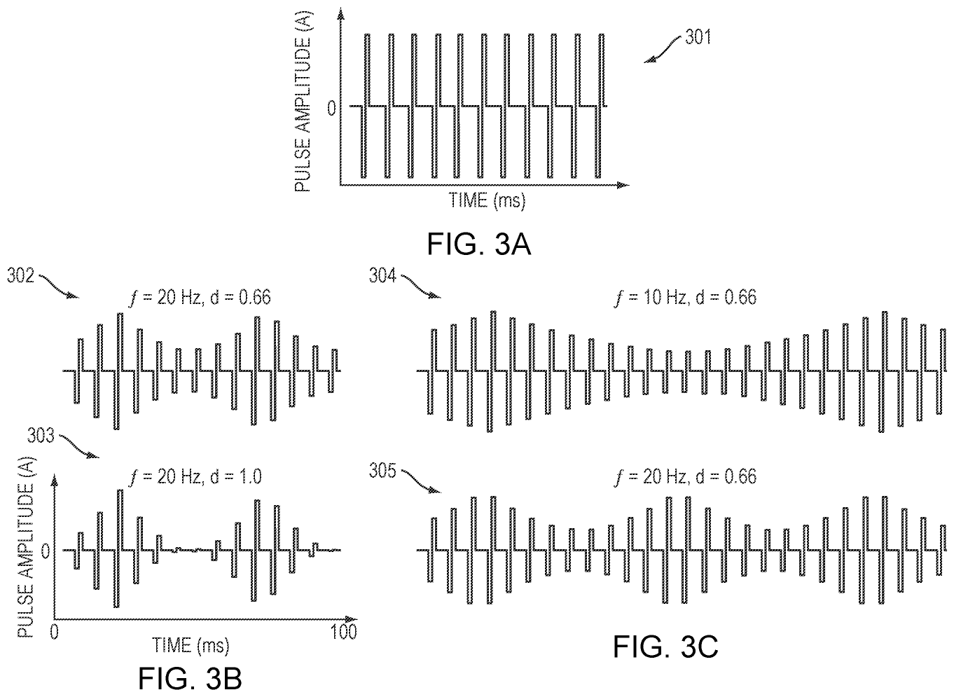

FIGS. 3A-C are timing diagrams illustrating example waveform profiles that may be output by the pulse generator 105. FIG. 3A illustrates a waveform profile 301 having a series of identical pulses at a fixed frequency (typically, 130-185 pulses per second), and the pulse settings (amplitude, shape, duration) are constant and programmed in advanced. This profile 301 exemplifies an electrical waveform that has been approved by the United States Food and Drug Administration (FDA) for administration of DBS therapy.

Typical DBS therapies involve the generation and delivery of electric pulses that are equal in amplitude and shape and are set at equal intervals, as exemplified by the profile 301 of FIG. 3. In a typical configuration, a user programs the DBS therapy by selecting the amplitude and frequency of the pulses, wherein the frequency sets the duration of the inter-pulse intervals. Stimulation parameters, such as amplitude and frequency, are kept constant between programming sessions.

Example embodiments can provide for DBS therapy matching the therapeutic efficacy of current, regular DBS while extending battery life by limiting the amount of electrical stimulation delivered over time. Such examples may develop novel electrical stimulation patterns for use in DBS applications. These temporal patterns may modulate the amplitude of electrical stimuli over time per pre-defined profiles. Such embodiments may be leveraged in existing DBS systems or implemented in new systems/hardware, and, thus, may potentially encompass computer-implemented methods, software, and hardware.

Previous studies of DBS therapies indicate that, while DBS therapies other than regular DBS can be effective in treating movement disorders, the formulation of such DBS therapies is a challenging problem, the solution to which cannot be derived from current DBS design techniques. Furthermore, these studies demonstrate the sensitivity of neurological disorders such as essential tremor and Parkinson's disease to changes in the temporal arrangement of the DBS pulses. Example embodiments, in contrast, provide for modulation of the pulse amplitude rather than the optimization of the temporal arrangement of the DBS pulses.

Example embodiments, such as the system 100, may implement open-loop DBS therapies wherein the amplitude of the electric pulses is varied over time according to a prerecorded function referred to as an amplitude modulation envelope. An amplitude modulation envelope may define the changes in the amplitude of an electrical signal over time. The envelope may be a periodic function with a fixed duration T>0 that is either preconfigured or set by a user, and the values of this function over one period may be generated by the controller 120 in the neurostimulator or stored in a memory buffer (e.g., parameter data store 125) within the pulse generator 105.

FIGS. 3B and 3C illustrate example waveform profiles 302-305 that may be implemented by the controller 120 to generate the signal waveform 142 described above. Embodiments that employ amplitude-modulated DBS (AM-DBS) may generate pulses that have the same shape and duration, and are equally spaced, while the pulse amplitude varies in time according to a pre-selected rule that is programmed in the pulse generator 105. For example, the pulse amplitude Aₖ of the generic k-th pulse at the generic time tₖ may vary according to the rule:

$${A}_{k} = A [ ( 1 - \frac{d}{2} ) + \frac{d}{2} sin ( 2 π {ft}_{k} ) ]$$

where A is the maximum amplitude (e.g., an FDA-approved DBS modality), and parameters d (modulation depth) and f (envelope frequency) are programmed in advance to tailor the pulse stimulation to the patient's actual needs. FIG. 3B shows waveform profiles 302-303 obtained with envelope frequency f=20 Hz, wherein profile 302 exhibits amplitude modulation with 66% depth (d=0.66), and profile 303 exhibits amplitude modulation with 100% depth (d=1.0). FIG. 3C shows waveform profiles 304-305 obtained with modulation depth d=0.66, wherein profile 304 exhibits amplitude modulation applied under an envelope of frequency f=10 Hz, and profile 305 exhibits amplitude modulation applied under an envelope of frequency f=20 Hz.

In a further embodiment, the amplitude A of DBS pulses may be varied over time t based on a generative rule such as the following equation:

A(t)=A(1+d sin(ωt))

wherein the parameter d may determine the depth of the pulses (i.e., amplitude modulation as exemplified by profiles 302-303) and parameter ω may determine how often the amplitude pattern is repeated (“frequency modulation”). The controller 120 may generate the pulses (e.g., pulses of the signal waveform 142) based on one of the profiles 302-305, or based on patterns obtained by combining an amplitude modulation profile such as the profiles 302-303 with a frequency modulation profile such as the profiles 304-305. The controller 120 can further optimize performance by adjusting the frequency of the envelope function (ω) and the depth of the modulation (d).

Embodiments that employ AM-DBS, as described above, may entrain neurons in a common oscillation at the envelope frequency f, which may support the suppression of ongoing pathological oscillations that are prevalent in patients with neurological diseases (e.g., Parkinson's Disease [PD]) and that are also well-correlated with clinical symptoms. Thus, the underlying mechanism of AM-DBS is different from currently FDA-approved DBS (“regular DBS”). Moreover, in many applications, amplitude-modulated electrical pulses may recruit neurons more effectively than constant-amplitude pulses. A further advantage of example embodiments is backward-compatibility to existing and FDA-approved devices, as such devices may require only minor hardware updates, and can be programmed with a software update to implement AM-DBS operation as described herein. Example embodiments may reduce consumption of an implanted battery by the pulse generator and may be implemented in multiple commercial systems.

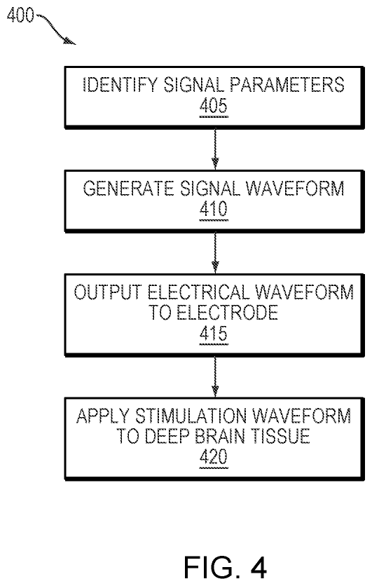

FIG. 4 is a flow diagram illustrating an example process 400 of implementing DBS. The process 400 may be implemented by the DBS system 100 described above. With reference to FIGS. 1-3, upon activating the pulse generator 105 and/or initializing a DBS session (e.g., via an input at the external controller 180), the controller 120 may first identify the signal parameters for the signal waveform 142 (405). The signal parameters may be previously determined during a programming phase of the system 100, wherein a trained physician may determine an appropriate maximum amplitude, frequency, and/or pulse width of the electrical pulses to be delivered by system 100. A trained physician may also determine the envelope function and the values of the envelope frequency f and depth d for AM-DBS. Such characteristics may be recorded and stored, as the signal parameters, to the parameter data store 125. For example, the controller 120 may store/retrieve at the parameter data store 125 one or more different signal parameters for generating signal waveforms to the parameter that results in a stimulation waveform having characteristics that treat one or more pathological conditions, such as Parkinson's disease (PD), essential tremor (ET), obsessive compulsive disorder (OCD), depression, epilepsy, or psychiatric disorders such as post-traumatic stress disorder (PTSD). Such signal parameters may be previously loaded to the parameter data store 125 during the programming phase and/or prior to initialization of the DBS session. Alternatively, the controller 120 may receive signal parameters from the external controller 180 via the communications interface 122. The controller 120 may also identify the signal parameters based on user input, such as a selection by the patient 190 at the external controller 180. Such a selection may include a given DBS treatment, which is associated with a particular signal waveform defined by an envelope function and one or more function generation parameters.

The controller 120 may then generate the signal waveform 142 having a signal profile (e.g., profiles 302-305) in accordance with the signal parameters (410). The signal waveform 142 may define a series of DBS pulses within an amplitude modulation envelope function. The frequency of the amplitude modulation envelope may be less than or equal to half of a frequency of the DB S pulses. For example, the controller may generate the DBS pulses with a frequency of about 50 Hertz to about 10,000 Hertz and generate the amplitude modulation envelope with a frequency of about 1 Hertz to about 5,000 Hertz. DBS pulses with a frequency of about 130 Hertz to about 185 Hertz, in combination with an amplitude modulation envelope with a frequency of about 1 Hertz to about 92.5 Hertz, may be particularly effective in treating a variety of pathological conditions. The controller 120 may generate the amplitude modulation envelope at a maximum amplitude and frequency that entrains the respective neurons of the region of deep brain tissue 194, at which the DBS electrode 110 is located, in the normal oscillation pattern in the region of deep brain tissue 194. The controller 120 may further adjust the amplitude modulation envelope of the pulses with a pre-programmed variation in amplitude that entrains the respective neurons of the region of deep brain tissue 194 in the normal oscillation pattern. For example, the controller 120 may adjust the amplitude modulation envelope from about 1 percent depth to 100 percent depth.

The driver 124 may receive the signal waveform, generate a corresponding electrical waveform 144, and transmit the electrical waveform 144 to the DBS electrode 110 via the lead 107 (415). The DBS electrode 110, in turn, may convert the electrical waveform to a stimulation waveform, and apply the stimulation waveform to the deep brain tissue 194 of the patient 190 (420). Further, based on information about the region of deep brain tissue 194 at which the DBS electrode 110 is located, the controller 120 may adjust the signal waveform under parameters to cause the DBS electrode 110 to produce a stimulation waveform specific to the respective region of deep brain tissue 194. As described above, the DBS electrode 110 may be implanted in electrical communication with the deep brain tissue 194, which has been determined to exhibit a pathological oscillation pattern related to the pathological condition. By applying the stimulation waveform to the deep brain tissue 194, the DBS electrode 110 may entrain neurons of the deep brain tissue 194 in a normal oscillation pattern, thereby suppressing the pathological oscillation pattern to treat the pathological condition.

The controller 120 may also adjust the signal waveform 142, and, thus, the stimulation applied by the DBS electrode 110, based on one or more inputs. For example, a clinician or the patient 190 may interface with the external controller 180 to adjust stimulation programming depending on feedback of the patient's reaction to treatment with the stimulation waveform. This feedback may include the patient's 190 subjective experience during the DBS session, observation of the patient 190 by the clinician, and/or feedback collected by the sensor 112 and presented at the external controller 180. Alternatively, the controller 120 may automatically adjust the signal waveform based on feedback collected by the sensor 112 (e.g. a measurement indicating a response of the deep brain tissue 194 during the DBS session) and transmitted via the communications interface 122.

The controller 120 may also provide additional functionality utilizing the sensor 112. For example, the controller 120 may provide data from the sensor 112, or information based on the data, to a clinician or the patient 190 via the external controller 180 (e.g., a display, touchscreen, or other interface). The controller 120 may adjust a parameter of the amplitude modulation envelope of the signal waveform 142 as a function of the data received from the sensor 112. For example, the controller 120 may adjust the signal waveform 142 in response to detecting, via the sensor 112, that the presently-applied stimulation waveform is ineffective in entraining the deep brain tissue 194 to treat the pathological condition, or that the stimulation waveform is providing excessive stimulation to the deep brain tissue 194.

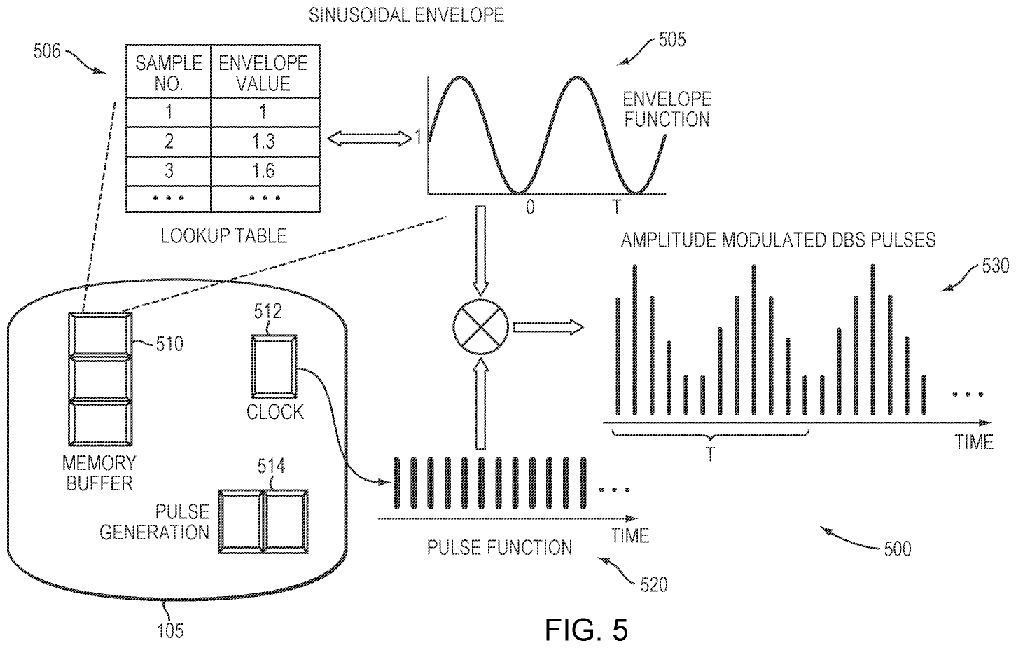

FIG. 5 is a diagram illustrating a process 500 of generating a signal waveform for one phase of pulses in one embodiment. The process 500 may be completed by the DBS system 100 described above. Here, the envelope is a sinusoidal function that may be stored at the parameter data store 125. The mathematical representation of the envelop 505 may be discretized in a lookup table 506 that is allocated in a memory buffer 510 within the pulse generator 105. The pulse generator 105 may be configured with hardware and software to determine the timing of each DBS pulse 530, e.g., using an internal clock 512, thereby generating a pulse function 520. For each DBS pulse, the amplitude may be determined by the value of the envelope at the time of the pulse, and the pulse generator 105 may generate the electric pulse at the amplitude determined by the envelope by combining the envelope function 505 and the pulse function 520. If the time of a pulse exceeds the duration T of the envelop 505 stored in the lookup table 506, the amplitude of the pulse may be determined by computing the time modulo T (530).

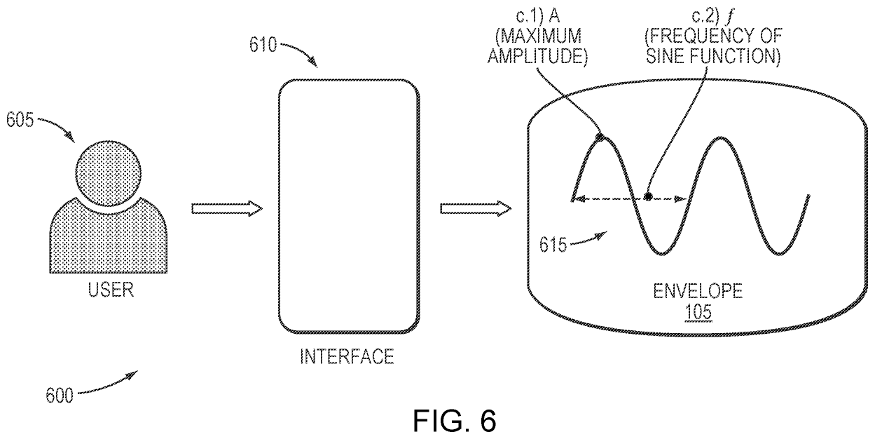

FIG. 6 is a diagram illustrating a process 600 of user adjustment of a signal waveform. A user 605 (e.g., a clinician or a patient) can use an interface 610 (e.g., the external controller 180) to change the shape of a sinusoidal envelop 615 generated by the pulse generator 615. The interface 610 may include a remote controller, a computer program, and/or a software application for mobile devices. The shape of the envelop 615 may be manipulated by changing model parameters for the envelope function. These parameters can include the maximum value A of the envelop (c.1) and the frequency f of the sine (c.2).

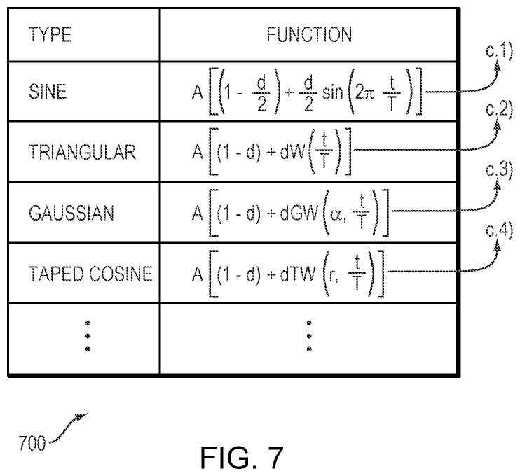

FIG. 7 illustrates a function lookup table 700 that may be stored at the pulse generator 105 at the parameter data store 125 or another location. Referring to FIG. 6, the user 605 can use the interface 610 to select the envelope function among a preconfigured menu of functions corresponding to the table 700, where functions may be generated by different models. The list of models may include:
a) Sinusoidal functions of period T>0, maximum amplitude A, and depth d (c.1), where parameters A, T, and d can be selected by the user using the interface (b).
b) Triangular windows of duration T>0, maximum amplitude A, and depth d (c.2), where parameters A, T, and d can be selected by the user using the interface (b). Windows are repeated (e.g., every T) and result in a periodic function. Symbol “W” in (c.2) denotes the function:

$$W ( \frac{t}{T} ) = \{ \begin{matrix}\frac{2 t}{T} , 0 \leq t < \frac{T}{2} \\ 2 - \frac{2 t}{T} , \frac{T}{2} \leq t < T\end{matrix}$$

c) Gaussian windows of duration T>0, maximum amplitude A, and depth d (c. 3), where parameters A, T, and d can be selected by the user using the interface (b). Parameters such as α in (c.3) can also be programmed. Windows are repeated (e.g., every T) and result in a periodic function. Symbol “GW” in (c.3) denotes the function:

$$G W ( α , \frac{t}{T} ) = exp [ - 2 {( α \frac{t}{T} )}^{2} ] .$$

d) Taped cosine windows (a.k.a., “Tukey” windows) of duration T>0, maximum amplitude A, and depth d (c.4), where parameters A, T, and d can be selected by the user using the interface (b). Parameters such as r in (c.4) can also be programmed. Windows are repeated (e.g., every T) and result in a periodic function. Symbol “TW” in (c.4) denotes the function:

$$T W ( r , \frac{t}{T} ) = \{ \begin{matrix}\frac{1}{2} \{ 1 + cos ( \frac{2 π}{r} [ \frac{t}{T} - \frac{r}{2} ] ) \} , & 0 \leq \frac{t}{T} < \frac{r}{2} \\ 1 , & \frac{r}{2} \leq \frac{t}{T} < 1 - \frac{r}{2} \\ \frac{1}{2} \{ 1 + cos ( \frac{2 π}{r} [ \frac{t}{T} - 1 + \frac{r}{2} ] ) \} , & 1 - \frac{r}{2} \leq \frac{t}{T} < 1\end{matrix}$$

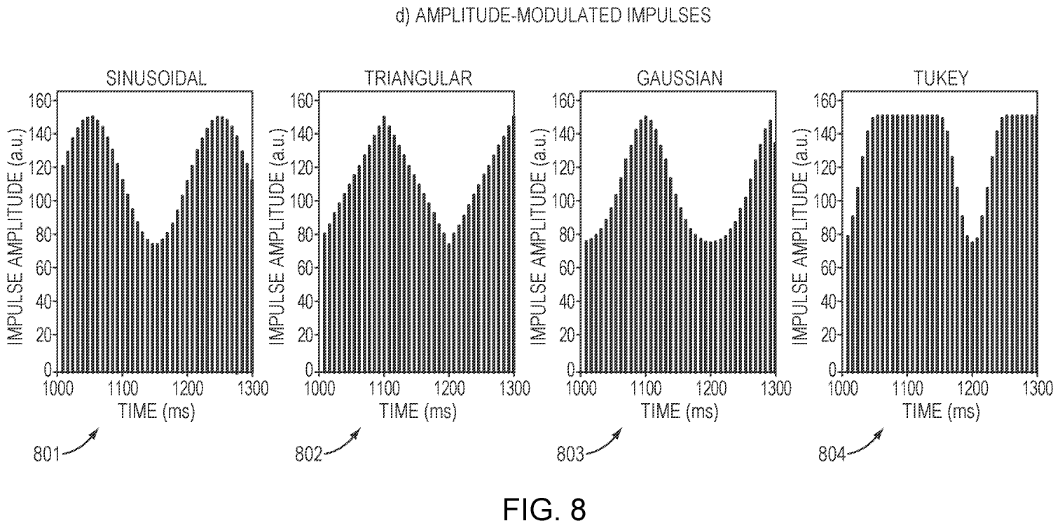

FIG. 8 illustrates the contours of the train of pulses (one pulse phase only is depicted) for example envelope functions belonging to the classes defined as sinusoidal 801, triangular 802, Gaussian 803, and Tukey 804. The envelope functions as depicted in FIG. 8 were obtained with parameters T=200 ms, A=150, d=0.5, α=3, and r=0.5. A total of 130 pulses per second were generated and depicted in panel d) as black vertical bars.

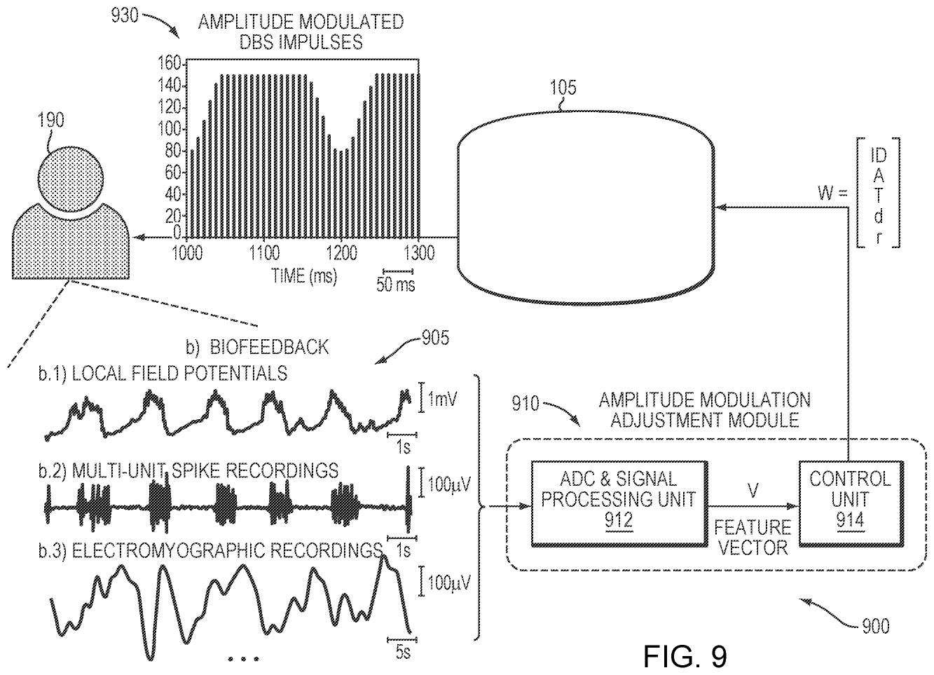

FIG. 9 is a diagram illustrating a process 900 of adjustment of a signal waveform based on biofeedback from the patient 190. Here, one or more biofeedback signals 905 are measured from the patient 190 via the sensor 112 and/or other suitable equipment. Biofeedback signals 905 may include local field potentials recorded in the patient's brain (b.1), multiunit spike recordings obtained from the DBS electrodes (b.2), and/or electromyographic recordings from the patient's muscles (b.3). The biofeedback signals may be fed to an amplitude modulation adjustment module 910, which may include hardware and software components such as an analog-to-digital converter (ADC), a single processing unit 912, and a control unit 914. The ADC and signal processing unit 912 may be used to calculate one or more biomarker features v of the disease, which can include measurements of power spectrum density, spectral coherence, temporal coherence, and amplitude-phase coupling between signals. The control unit 914 may implement one or more algorithms for the selection of the envelope model and the model parameters used to compute the amplitude modulated pulse sequence. The control unit 914 may provide a pointer (ID) to one envelope model function in a lookup table 700 (e.g., a Tukey function), along with the vector of parameters w used to generate the envelope, i.e., amplitude A, duration T, depth d, and saturation factor r. The amplitude modulation adjustment module 910 can either be a standalone module or a component of the pulse generator 105 (e.g., the controller 120). The pulse generator 105 may store a copy of the envelop lookup table 700 and implement the hardware and software necessary to generate the amplitude modulated electric pulses 930.

Several neurological conditions are currently treated with DBS. Depending on the type of disease or disorder, the use of DBS is either approved by FDA or currently under investigation. The following list and references therein provide a summary of the disorders for which DBS is indicated. Disorders are organized by clinical manifestations. Example embodiments may be implemented to treat any of the conditions described below:
a) Movement disorders: DBS therapies are approved by the FDA for the treatment of movement disorders in Parkinson's disease and essential tremor. The approved surgical targets are the subthalamic nucleus, the globus pallidus, and the thalamus (ventrolateral and intermediate portions). The same targets are approved under humanitarian device exemption (HDE) for dystonia. It is estimated that more than 150,000 patients affected by movement disorders have been treated with DBS thus far. Also, DBS therapies are currently under FDA consideration to treat tics secondary to the Tourette syndrome, with a proposed target of stimulation being the centromedial thalamus.
b) Psychiatric disorders: DBS therapies are approved by FDA under HDE for the treatment of obsessive-compulsive disorders. The approved surgical targets are the ventral capsule and the ventral striatum. Moreover, several clinical trials and preclinical studies are investigating the effects of DBS therapies on treatment-resistant depression (TRD) and post-traumatic stress disorders (PTSD). The following surgical targets have been tested or are currently under investigation for the treatment of TRD: subgenual anterior cingulate cortex, internal capsule, ventral capsule, ventral striatum, nucleus accumbens, and medial forebrain bundle. The following surgical targets are currently under investigation for the treatment of PTSD: basolateral nucleus of the amygdala, ventral striatum, hippocampus, and prefrontal cortex.
c) Epilepsy: DBS therapies are approved by FDA as add-on treatments for drug-resistant focal epilepsy. “Focal” epilepsy means that the patients have seizures with focal onset in the brain (a.k.a., “partial seizures”). The approved surgical target is the anterior nucleus of the thalamus. Furthermore, clinical trials are investigating alter-native targets, including the anterior and centromedial nuclei of thalamus, the subthalamic nucleus, the cerebellum, and the hippocampus. Finally, two additional neuromodulation therapies are approved by FDA for the treatment of epilepsy, i.e., vagus nerve stimulation (VNS) and responsive neurostimulation (RNS). Although these therapies considered alternative to DBS, they may deliver an amplitude modulated stimulus.
d) Stroke: At least one clinical trial, is ongoing and aims to assess the effects of DBS therapies during the rehabilitation of individuals with strokes. The approved surgical target for the DBS lead is the dentate nucleus of the cerebellum.

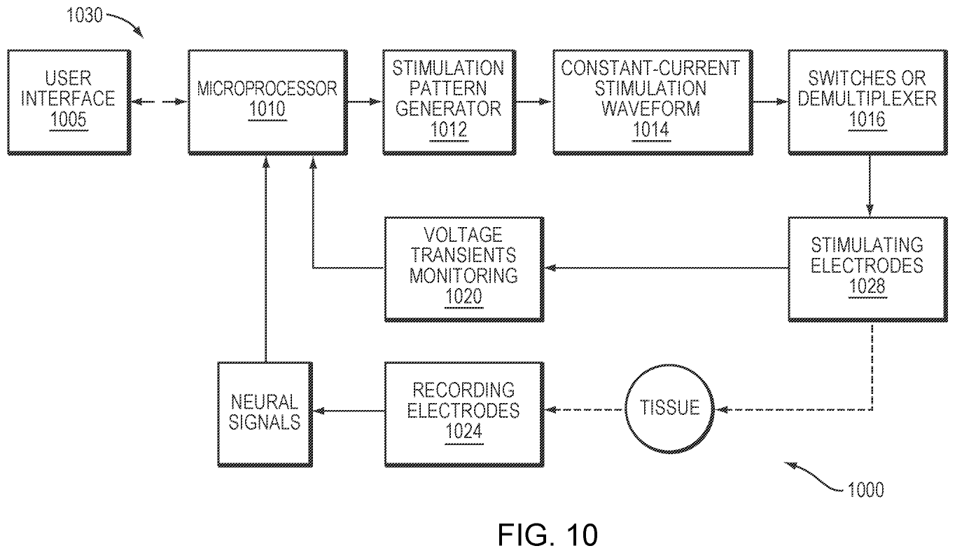

FIG. 10 is a functional block diagram of a DBS system 1000 in a further embodiment. The system 1000 may include a user interface 1005, a PCB board including a microprocessor 1010, a stimulation pattern generator 1012, a voltage-to-current conversion unit 1014, 16 SPST switches 1016, an evoked neural response recording unit 1024, a stimulus voltage waveform monitoring unit 1020, a Bluetooth interface 1030, and micro/bipolar electrodes 1028. The system 1000 may further incorporate some or all of the features of the DBS system 100 described above, and likewise, the DBS system 100 may incorporate some or all of the features of the DBS system 1000.

As depicted, the system 1000 integrates multiple channels (e.g., 16) of constant-current stimulation 1012 & 1014 and voltage monitors 1020, multiple channels (e.g., 2) of neural recording 1024, wireless communication (e.g., via Bluetooth 4.0) 1030, a power management unit, battery monitoring, and other passive components. The microprocessor 1010 can be programmed to generate the stimulation waveforms based on user inputs. For a biphasic voltage signal generation, the microprocessor creates digital codes for monophasic signals, which are, then, converted to analog biphasic signals using a DAC (Digital-to-Analog Converter) with a non-overlapping-two phase signal generation circuits. The data is transmitted from the microprocessor to the DAC via SPI (serial peripheral interface) bus over GPIO (general purpose input output) interface 1012. The voltage waveform of the signal is then converted into current form with a Howland current pump circuit 1014. The biphasic current signals are then directed to a group of electrodes 1028 by switches or a demultiplexer 1016. Voltage transients that result on the stimulating electrode are collected by a multiplexer, shifted up in values, and converted to digital signals by an ADC (analog-to-digital) converter 1020. This operation can also be done within microprocessor's built-in ADC circuits 1010. The digital voltage transient signals are then fed to the user interface 1005, wirelessly through the Bluetooth connection 1030, to monitor and record electrode polarization, and thus, to ensure safety of electrical stimulation. The electrodes 1028 may include more stimulating electrodes, for example, 1000 channels, with additional switches or demultiplexers 1016.

At least two channels were allocated for differential-recordings of evoked or spontaneous neural responses 1024. First, passive high-pass filters attenuated low-frequency components of the signals with a cut-off frequency of, for example, 10 Hz. The cut-off frequency can be varied depending on signals of interested. For example, if only spike activities of neurons are wanted, then the cut-off frequency can be set at 300 Hz. Second, the analog signals were differentiated and amplified. Third, the microcontroller on-chip analog-to-digital conversion (ADC) 1010 was performed. Finally, the digitized evoked neural response signals are sent back to the user interface 1005 through the Bluetooth connection 1030. There can be more than two channels for evoked or spontaneous neural recordings. If more recording electrode are added 1024, for example, 1000 channels, each recorded signal can be differentially-amplified against a ground electrode or against each other in the same manner. These circuits 1000 can be made with surface-mountable devices (SMDs) on PCBs (printed circuit boards) or by VLSI (very large scale integrated) circuits. In addition, with enclosed assembly of individual SMD components, the lead length can be short, which yields faster signal transmission among the peripherals.

The microprocessor such as ARM Cortex processor 1010 can be software-programmed. STM32CubeMX toolchain assigns each pin a specific function, such as for USART (Universal Synchronous/Asynchronous Receiver/Transmitter) communication. Once configured, the codes are generated in the uVision Keil MDK microcontroller develop kit which then provides environment for programming. The microprocessor 1010 may not require receiving commands at all times. Once stimulation settings are set, the microprocessor 1010 may run autonomously for a substantial length of time (e.g., many hours), thereby conserving battery power.

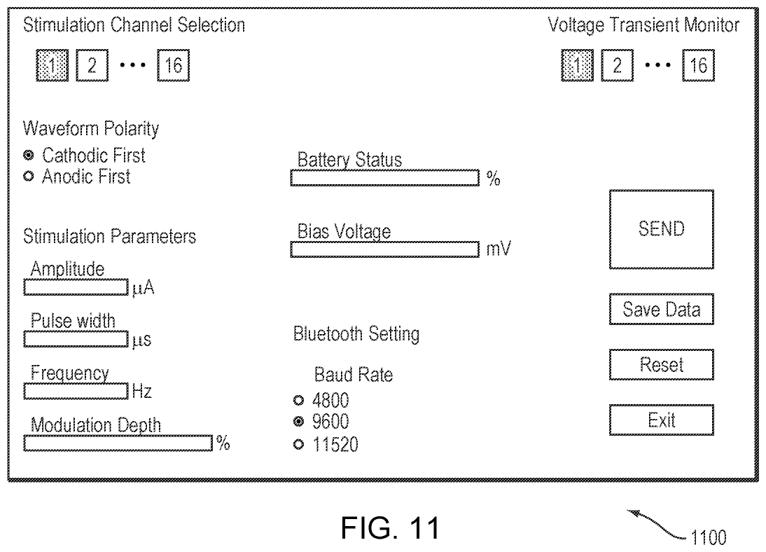

FIG. 11 illustrates a user interface 1100 enabling a user to monitor and control the DBS system 1000, and which may be implemented in the user interface 1005. The interface 1100 has been encoded and designed in Microsoft Visual Studio and has been programmed in Visual Basic platform. Major parts of the interface include Bluetooth communication panel a stimulation parameters panel, where the stimulation signal amplitude, pulse width, frequency, and bias voltage can be specified, stimulation channel selection and voltage transient monitoring panel, a stimulation polarity panel to select the first phase to be cathodic or anodic, battery monitor panel, and an amplitude modulation panel, where modulation depth of the signal can be adjusted. The interface 1005 wirelessly communicates with the microprocessor.

Example embodiments may include a computer program product, including a non-transitory computer-readable medium (e.g., a removable storage medium such as one or more DVD-ROM's, CD-ROM's, diskettes, tapes, etc.) that provides at least a portion of the software instructions for the invention system. The computer program product can be installed by any suitable software installation procedure, as is well known in the art. In another embodiment, at least a portion of the software instructions may also be downloaded over a cable communication and/or wireless connection. The computer program product may be transmitted via a propagation medium (e.g., a radio wave, an infrared wave, a laser wave, a sound wave, or an electrical wave propagated over a global network such as the Internet, or other network(s)). Such carrier medium or signals may be employed to provide at least a portion of the software instructions for routines/programs of example embodiments.

While example embodiments have been particularly shown and described, it will be understood by those skilled in the art that various changes in form and details may be made therein without departing from the scope of the embodiments encompassed by the appended claims.

1. A deep brain stimulation (DBS) system for treating a pathological condition, the DBS system comprising:
a controller configured to generate a signal waveform defining a series of DBS pulses within an amplitude modulation envelope, a frequency of the amplitude modulation envelope being less than or equal to half of a frequency of the DBS pulses;
a driver circuit including an input port and an output port, the driver circuit configured to amplify the signal waveform received from the controller via the input port to produce an electrical waveform available at the output port; and
at least one DBS electrode configured to be implanted in electrical communication with deep brain tissue within a brain of a patient, the deep brain tissue known to exhibit a pathological oscillation pattern related to the pathological condition, the at least one DBS electrode electrically coupled to the output port of the driver circuit and configured to convert the electrical waveform to a stimulation waveform to entrain neurons of the deep brain tissue in a normal oscillation pattern, thereby suppressing the pathological oscillation pattern to treat the pathological condition.

2. The DBS system of claim 1, wherein the at least one DBS electrode is configured to be implanted in a region of deep brain tissue selected from a group consisting of:
the hippocampus, amygdala, medial prefrontal cortex, orbitofrontal cortex, anterior cingulate cortex, striatum, including the putamen and the caudate nucleus, thalamus, including the anterior, ventrointermediate, ventrolateral and dorsolateral thalamus, basal ganglia, dentate nucleus or substantia nigra; and
wherein the controller is further configured to adjust the signal waveform with the amplitude modulation envelope having characteristics that cause the at least one DBS electrode to produce a stimulation waveform specific to the respective region of deep brain tissue at which the at least one DBS electrode is located.

3. The DBS system of claim 1, wherein the at least one DBS electrode is further configured to extend into the brain of the patient at a depth of about 1 cm to about 15 cm.

4. The DBS system of claim 1, wherein the controller is configurable to store parameters for generating the signal waveform that results in the stimulation waveform having characteristics that treat at least one of the following pathological conditions: Parkinson's disease (PD), essential tremor (ET), obsessive compulsive disorder (OCD), depression, epilepsy, or post-traumatic stress disorder (PTSD).

5. The DBS system of claim 1, wherein the controller is further configured to generate the amplitude modulation envelope at an amplitude and frequency that entrains the respective neurons of the region of deep brain tissue, at which the at least one DBS electrode is located, in the normal oscillation pattern in the region of deep brain tissue at which the at least one DBS electrode is located.

6. The DBS system of claim 1, wherein controller is further configured to adjust the amplitude modulation envelope of the pulses with a pre-programmed variation in amplitude that entrains the respective neurons of the region of deep brain tissue, at which the at least one DBS electrode is located, in the normal oscillation pattern.

7. The DBS system of claim 1, wherein the controller is further configured to adjust the amplitude modulation envelope from about 1 percent depth to 100 percent depth.

8. The DBS system of claim 1, wherein the controller is further configured to generate the DBS pulses with a frequency of about 50 Hertz to about 10,000 Hertz and to generate the amplitude modulation envelope with a frequency of about 1 Hertz to about 5,000 Hertz.

9. The DBS system of claim 1, wherein the controller is further configured to enable a clinician or the patient to adjust stimulation programming depending on feedback of the patient's reaction to treatment with the stimulation waveform.

10. The DBS system of claim 1, wherein the controller is further configured to define the amplitude modulation envelope based on a user input, the user input including at least one of a function and one or more function generation parameters.

11. The DBS system of claim 1, further comprising at least one sensor that is communicatively coupled to the controller,
wherein the at least one sensor is configured to be disposed in or at the patient at a location to observe an expression of an affectation that is symptomatic of the pathological condition; and
wherein the location to observe the expression of the affectation is selected from a group consisting of the region of deep brain tissue at which at least one of the at least one DBS electrode is located, a location elsewhere within the patient, or a location at which movement of the patient is observable.

12. The DBS system of claim 11, wherein the controller that is communicatively coupled to the at least one sensor is further configured to provide data from the at least one sensor, or information based on the data, to the clinician via an interface.

13. The DBS system of claim 11, wherein the controller is further configured to adjust a parameter of the amplitude modulation envelope as a function of data received from the at least one sensor.

14. The DBS system of claim 11, wherein the at least one sensor is further configured to be coupled to an appendage or head of the patient, offset from the patient with an orientation to observe motion of the patient, motion of an appendage of the patient, motion of a head of the patient, or offset from the patient with an orientation to detect eye gaze or eye saccades of the patient.

15. The DBS system of claim 11, wherein the location elsewhere within the patient is a subcutaneous, epidural, subdural, intracortical, or subcortical region of the patient.

16. The DBS system of claim 11, wherein the at least one sensor includes at least one of the following sensor types: an electro-chemical sensor, motion sensor, optical sensor, accelerometer, and imaging device.

17. The DBS system of claim 1, wherein the driver circuit is configured to operate as a stimulation pattern generator that generates voltage waveform and converts the signal waveform to a current-controlled electrical waveform.

18. A method of treating a pathological condition using deep brain stimulation (DBS), the method comprising:
generating a signal waveform defining a series of DBS pulses within an amplitude modulation envelope, a frequency of the amplitude modulation envelope being less than or equal to half of a frequency of the DBS pulses;
amplifying the signal waveform to produce a corresponding electrical waveform;
converting the electrical waveform to a stimulation waveform via at least one DBS electrode implanted in electrical communication with deep brain tissue within a brain of a patient, the deep brain tissue known to exhibit a pathological oscillation pattern related to the pathological condition; and
applying the stimulation waveform to the deep brain tissue to entrain neurons of the deep brain tissue in a normal oscillation pattern, thereby suppressing the pathological oscillation pattern to treat the pathological condition.

19. The method of claim 18, wherein the at least one DBS electrode is configured to be implanted in a region of deep brain tissue selected from a group consisting of:
the hippocampus, amygdala, medial prefrontal cortex, orbitofrontal cortex, anterior cingulate cortex, striatum, including the putamen and the caudate nucleus, thalamus, including the anterior, ventrointermediate, ventrolateral and dorsolateral thalamus, basal ganglia, dentate nucleus, fornix, or substantia nigra; and
further comprising adjusting the signal waveform with the amplitude modulation envelope having characteristics that cause the at least one DBS electrode to produce a stimulation waveform specific to the respective region of deep brain tissue at which the at least one DBS electrode is located.

20. The method of claim 18, wherein the at least one DBS electrode is further configured to extend into the brain of the patient at a depth of about 1 cm to about 15 cm.

21. The method of claim 18, further comprising storing parameters for generating the signal waveform that results in the stimulation waveform having characteristics that treat at least one of the following pathological conditions: Parkinson's disease (PD), essential tremor (ET), obsessive compulsive disorder (OCD), depression, epilepsy, or post-traumatic stress disorder (PTSD).

22. The method of claim 18, further comprising generating the amplitude modulation envelope at an amplitude and frequency that entrains the respective neurons of the region of deep brain tissue, at which the at least one DBS electrode is located, in the normal oscillation pattern in the region of deep brain tissue at which the at least one DBS electrode is located.

23. The method of claim 18, further comprising adjusting the amplitude modulation envelope of the pulses with a pre-programmed variation in amplitude that entrains the respective neurons of the region of deep brain tissue, at which the at least one DBS electrode is located, in the normal oscillation pattern.

24. The method of claim 18, further comprising adjusting the amplitude modulation envelope from about 1 percent depth to 100 percent depth.

25. The method of claim 18, further comprising generating the DB S pulses with a frequency of about 50 Hertz to about 10,000 Hertz and to generate the amplitude modulation envelope with a frequency of about 1 Hertz to about 5,000 Hertz.

26. The method of claim 18, further comprising enabling a clinician or the patient to adjust stimulation programming depending on feedback of the patient's reaction to treatment with the stimulation waveform.

27. The method of claim 18, further comprising defining the amplitude modulation envelope based on a user input, the user input including at least one of a function and one or more function generation parameters.

28. The method of claim 18, further comprising retrieving data from at least one sensor that is communicatively coupled to the controller, wherein the at least one sensor is configured to be disposed in or at the patient at a location to observe an expression of an affectation that is symptomatic of the pathological condition; and
wherein the location to observe the expression of the affectation is selected from a group consisting of the region of deep brain tissue at which at least one of the at least one DBS electrode is located, a location elsewhere within the patient, or a location at which movement of the patient is observable.

29. The method of claim 28, further comprising providing data from the at least one sensor, or information based on the data, to the clinician via an interface.

30. The method of claim 28, further comprising adjusting a parameter of the amplitude modulation envelope as a function of data received from the at least one sensor.

31. The method of claim 28, wherein the at least one sensor is further configured to be coupled to an appendage or head of the patient, offset from the patient with an orientation to observe motion of the patient, motion of an appendage of the patient, motion of a head of the patient, or offset from the patient with an orientation to detect eye gaze or eye saccades of the patient.

32. The method of claim 28, wherein the location elsewhere within the patient is a subcutaneous, epidural, subdural, intracortical, or subcortical region of the patient.

33. The method of claim 28, wherein the at least one sensor includes at least one of the following sensor types: an electro-chemical sensor, motion sensor, optical sensor, accelerometer, and imaging device.

34. The method of claim 18, further comprising converting the signal waveform to a current-controlled electrical waveform.

35. A computer-readable medium storing instructions that, when executed by a computer, cause the computer to:
generate a signal waveform defining a series of DBS pulses within an amplitude modulation envelope, a frequency of the amplitude modulation envelope being less than or equal to half of a frequency of the DBS pulses;
amplify the signal waveform to produce a corresponding electrical waveform; convert the electrical waveform to a stimulation waveform via at least one DBS electrode implanted in electrical communication with deep brain tissue within a brain of a patient, the deep brain tissue known to exhibit a pathological oscillation pattern related to the pathological condition; and
apply the stimulation waveform to the deep brain tissue to entrain neurons of the deep brain tissue in a normal oscillation pattern, thereby suppressing the pathological oscillation pattern to treat the pathological condition.
